(19) United States
(12) Patent Application Publication
(10) Pub. No.: US 2023/0135432 A1
(43) Pub. Date: May 4, 2023

(54) POLYMERIC NANOPARTICLES AND DERIVATIVES THEREOF FOR NUCLEIC ACID BINDING AND DELIVERY

(71) Applicant: University of Massachusetts, Boston, MA (US)

(72) Inventor: Sankaran Thayumanavan, Amherst, MA (US)

(21) Appl. No.: 18/088,544

(22) Filed: Dec. 24, 2022

Related U.S. Application Data
(62) Division of application No. 16/312,572, filed on Dec. 21, 2018.
(60) Provisional application No. 62/353,629, filed on Jun. 23, 2016.

(51) Int. Cl. A61K 47/58; A61P 35/00; C12N 15/87; C08F 220/38; C08F 220/28; A61K 9/06; A61K 31/713

(52) U.S. Cl. CPC A61K 47/58; A61P 35/00; C12N 15/87; C08F 220/385; C08F 220/286; A61K 9/06; A61K 31/713; B82Y 5/00

(57) ABSTRACT

The invention provides polymers and polymeric nanogels in which nucleic acid molecules can be stably entrapped or encapsulated and are controllably delivered and released upon degradation of the nano-structures in response to specific microenvironment triggers, and compositions and methods of preparation and use thereof.

### PRIORITY CLAIMS AND RELATED PATENT APPLICATIONS

This application claims the benefit of priority from U.S. Provisional Application Ser. No. 62/353,629, filed on Jun. 23, 2016, the entire content of which is incorporated herein by reference in its entirety.

### STATEMENT REGARDING FEDERALLY FUNDED RESEARCH OR DEVELOPMENT

This invention was made with government support under Grant No. W911NF-15-1-0568 and W911NF-13-1-0187 awarded by the U. S. Army Research Office. The Government has certain rights in the invention.

### TECHNICAL FIELDS OF THE INVENTION

The invention generally relates to polymers and polymer-based nano-structures. More particularly, the invention relates to polymers and polymeric nanogels to which nucleic acid molecules can stably bind and be controllably delivered and released upon degradation of the nano-structures in response to specific microenvironment, and compositions and methods of preparation and use thereof.

### BACKGROUND OF THE INVENTION

Recent years have seen fast increasing interests in nucleic acid-based technologies, such as RNA interference or “RNAi”, a powerful tool to target and silence specific gene expression. (Fire et al., 1998 Nature 391:806-811.) Double-stranded RNAs (dsRNAs) can provoke gene silencing in numerous in vivo contexts. Small interfering RNA (siRNA) and microRNA hold great promises as therapeutics of diversified human diseases. Similarly, mRNA based therapy is being considered as a powerful approach for treatment of many genetic disorders.

The clinical application of RNAi has been hindered by the lack of a delivery system that is safe, stable, and efficient. Various delivery systems have been studied, for example, viral vectors, cationic liposomes, cell-penetrating peptides (CPPs) and cationic polymers. (Tseng et al. 2009 Advanced Drug Delivery Reviews 61(9):721-731; Lewis et al. 2007 Advanced Drug Delivery Reviews 59(2-3):115-123.)

Significant limitations are encountered when using viral vectors, including issues associated with immunogenicity and inflammation. Cationic liposomes and cationic lipids and lipid-like materials, while being widely used for in vitro studies, present significant toxicity and efficiency restrains for in vivo applications. Similarly, approaches using cell penetrating peptides (CPP) have been taken. For the CPP-based approaches, the formation of nucleic acid bioconjugates with CPPs or CPP is driven by weak noncovalent interactions. As a result, these particles are usually unstable, particularly against serum nucleases leading to degradation and poor targeting of the RNA.

In cationic-polymer-based deliveries, siRNAs are assembled with cationic polymers through the electrostatic interactions. As in the case of the CPPs-based approach, such delivery systems tend to be unstable and prematurely dissociate and release siRNA before reaching the cytoplasm of the target cells.

Accordingly, an ongoing need remains for an effective delivery vehicle for RNA interference, one that is highly robust and effective and at the same time with low toxicity and long intracellular half-life enabling practical therapeutic applications.

### SUMMARY OF THE INVENTION

The present invention is based in part of the unexpected discovery of an effective delivery vehicle for nucleic acids (e.g., microRNA, mRNA, siRNA, plasmid DNA, and aptamers). The disclosed nucleic acid delivery system is highly robust and effective while characterized by low toxicity and long intracellular half-life, features essential for therapeutic applications. Importantly, the polymers, polymeric nanogels and nucleic acid delivery vehicles of the invention are readily prepared via simple and reliable synthetic techniques.

In one aspect, the invention generally relates to a crosslinked polymeric nanogel-nucleic acid assembly, comprising:
a polymeric nanogel comprising a block or random co-polymer comprising structural units of:

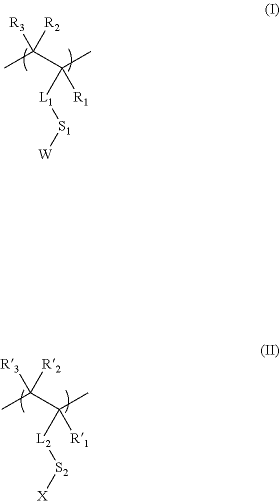

wherein
each of R₁ and R′₁ is independently a hydrogen, C₁-C₁₂ alkyl group, or halogen;
each of R₂, R′₂, R₃, and R′₃ is independently a hydrogen, (C₁-C₁₆) alkyl, (C₁-C₁₆) alkyloxy, or halogen;
each of L₁ and L₂ is independently a linking group;
each of S₁ and S₂ is independently a single bond or a spacer group;
W is a hydrophilic group; and
X is a group comprising a crosslinking moiety, and
a nucleic acid molecule entrapped or encapsulated in the polymeric nanogel.

In another aspect, the invention generally relates to a block or random co-polymer, having the structural formula:

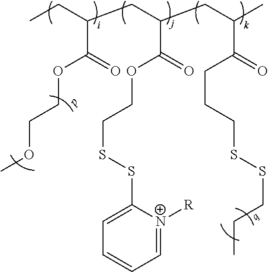

wherein
R is a C₁-C₁₅ alkyl group;
each of p and q is an integer from about 1 to about 20; and
each of i and j is independently a positive number, k may be zero or a positive number.

In yet another aspect, the invention generally relates to a method for delivering a nucleic acid molecule. The method includes: forming a crosslinked polymeric nanogel-nucleic acid assembly comprising a crosslinked polymeric nanogel and entrapped nucleic acid molecules therein, wherein the crosslinked polymeric nanogel is characterized by a polymeric network that is partially or completely free of cationic moieties; and directing the crosslinked polymeric nanogel-nucleic acid assembly to a target site.

### BRIEF DESCRIPTION OF THE DRAWINGS

FIG. 2 schematically illustrates any embodiment of the invention.

FIG. 3 shows an illustrative scheme for methylation of PEG-PDS copolymer and a ¹H NMR spectra before and after methylation.

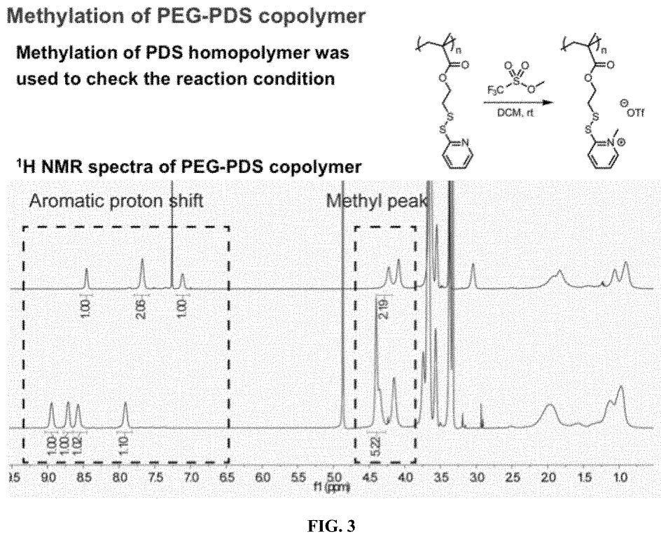

FIG. 4 shows increased positive charge density and better binding and crosslinking.

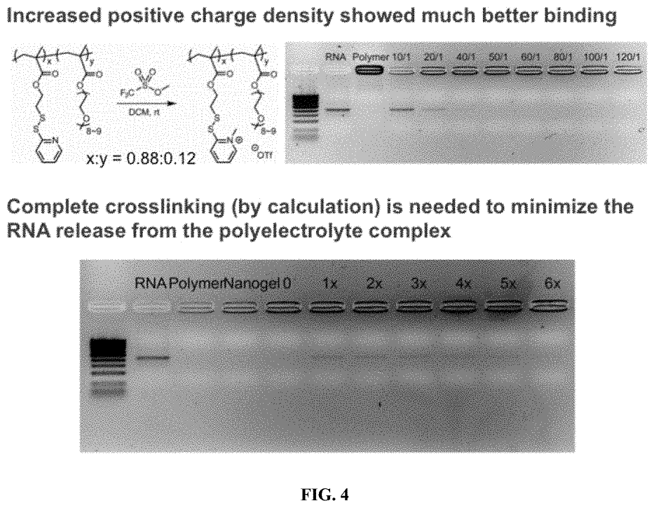

FIG. 5 shows an exemplary ¹H NMR spectrum of P2.

FIG. 6 shows an exemplary ¹H NMR spectrum of methylated P2.

FIG. 7 shows an exemplary ¹H NMR spectrum is P3.

FIG. 8 shows an exemplary ¹H NMR spectrum is methylated P3.

FIG. 9 shows an exemplary ¹H NMR spectrum is P4.

FIG. 10 shows an exemplary ¹H NMR spectrum is methylated P4.

FIG. 11 shows an exemplary Agarose gel electrophoresis of methylated P4

FIG. 12 shows an exemplary DTT-induced crosslinking.

FIG. 13 shows an exemplary dynamic light scattering and zeta potential measurement of P4.

FIG. 14 shows an exemplary crosslinking percentage in the presence of glutathione.

FIG. 15 shows an exemplary blastocyst development monitored at different preimplantation stages.

### DEFINITIONS

Definitions of specific functional groups and chemical terms are described in more detail below. General principles of organic chemistry, as well as specific functional moieties and reactivity, are described in “Organic Chemistry”, Thomas Sorrell, University Science Books, Sausalito: 2006. It will be appreciated that the compounds, as described herein, may be substituted with any number of substituents or functional moieties.

As used herein, “Cₓ-C_{y}” refers in general to groups that have from x to y (inclusive) carbon atoms. Therefore, for example, C₁-C₆ refers to groups that have 1, 2, 3, 4, 5, or 6 carbon atoms, which encompass C₁-C₂, C₁-C₃, C₁-C₄, C₁-C₅, C₂-C₃, C₂-C₄, C₂-C₅, C₂-C₆, and all like combinations. “C₁-C₁₅”, “C₁-C₂₀” and the likes similarly encompass the various combinations between 1 and 20 (inclusive) carbon atoms, such as C₁-C₆, C₁-C₁₂, C₃-C₁₂ and C₆-C₁₂.

As used herein, the term “alkyl”, refers to a hydrocarbyl group, which is a saturated hydrocarbon radical having the number of carbon atoms designated and includes straight, branched chain, cyclic and polycyclic groups. The term “hydrocarbyl” refers to any moiety comprising only hydrogen and carbon atoms. Hydrocarbyl groups include saturated (e.g., alkyl groups), unsaturated groups (e.g., alkenes and alkynes), aromatic groups (e.g., phenyl and naphthyl) and mixtures thereof.

As used herein, the term “Cₓ-C_{y} alkyl” refers to a saturated linear or branched free radical consisting essentially of x to y carbon atoms, wherein x is an integer from 1 to about 10 and y is an integer from about 2 to about 20. Exemplary Cₓ-C_{y} alkyl groups include “C₁-C₂₀ alkyl,” which refers to a saturated linear or branched free radical consisting essentially of 1 to 20 carbon atoms and a corresponding number of hydrogen atoms. Exemplary C₁-C₂₀ alkyl groups include methyl, ethyl, n-propyl, isopropyl, n-butyl, isobutyl, dodecanyl, etc.

As used herein, the term, “Cₓ-C_{y} alkoxy” refers to a straight or branched chain alkyl group consisting essentially of from x to y carbon atoms that is attached to the main structure via an oxygen atom, wherein x is an integer from 1 to about 10 and y is an integer from about 2 to about 20. For example, “C₁-C₂₀ alkoxy” refers to a straight or branched chain alkyl group having 1-20 carbon atoms that is attached to the main structure via an oxygen atom, thus having the general formula alkyl-O-, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

As used herein, the term “halogen” refers to fluorine (F), chlorine (Cl), bromine (Br), or iodine (I).

### DETAILED DESCRIPTION OF THE INVENTION

The present invention provides an effective delivery vehicle for nucleic acids. The nucleic acid delivery system disclosed herein is highly robust and effective and at the same time with low toxicity and long intracellular half-life enabling practical therapeutic applications. In addition, the polymers, polymeric nanogels and nucleic acid delivery vehicles of the invention can be prepared via simple and reliable synthetic techniques.

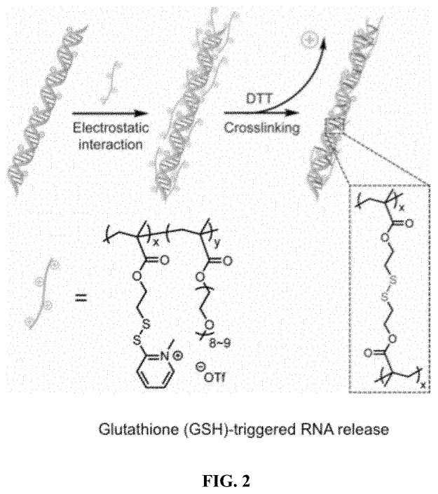

Methylation of the PDS moieties of the polymers enables microRNAs' binding to the polymer network leading to the formation of the nonagels. (FIG. 2) After the microRNAs binding and formation of the nanogels, the methylated, cationic PDS moieties are used to crosslink the nanogels and trap the microRNAs inside. In this process, the cationic charges are removed from the polymer, while still being able to lock up the microRNAs. As a result, a non-cationic and non-toxic delivery vehicle is achieved.

As disclosed herein, studies on the system with blastocysts has demonstrated that the system is an effective and promising approach to microRNA delivery, and nucleic acid delivery in general.

In one aspect, the invention generally relates to a crosslinked polymeric nanogel-nucleic acid assembly, comprising:
a polymeric nanogel comprising a block or random co-polymer comprising structural units of:

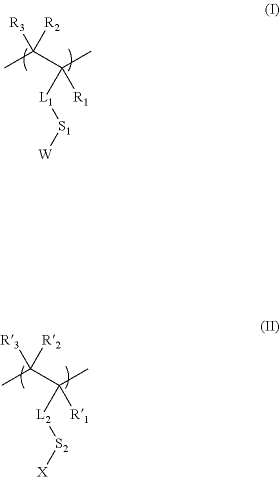

wherein
each of R₁ and R′₁ is independently a hydrogen, C₁-C₁₂ alkyl group, or halogen;
each of R₂, R′₂, R₃, and R′₃ is independently a hydrogen, (C₁-C₁₆) alkyl, (C₁-C₁₆) alkyloxy, or halogen;
each of L₁ and L₂ is independently a linking group;
each of S₁ and S₂ is independently a single bond or a spacer group;
W is a hydrophilic group; and
X is a group comprising a crosslinking moiety, and
a nucleic acid molecule entrapped or encapsulated in the polymeric nanogel.

In certain embodiments, the block or random co-polymer further comprises the structural unit of:

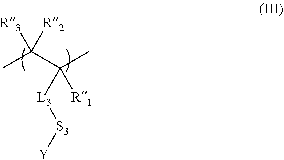

wherein
R″₁ is a hydrogen, C₁-C₁₂ alkyl group, or halogen;
each of R″₂ and R″₃ is independently a hydrogen, (C₁-C₁₆) alkyl, (C₁-C₁₆) alkyloxy, or halogen;
L₃ is a linking group;
S₃ is a single bond or a spacer group; and
Y is a non-crosslinking group.

In certain embodiments, X includes a crosslinked group.

In certain embodiments, X includes a group capable of forming a crosslinking bond.

In certain embodiments, the nucleic acid molecule is selected from single-stranded or double-stranded types. In certain embodiments, the nucleic acid molecule is selected from the group consisting of siRNA, microRNA, mRNA, ncRNA, catalytic RNA, guide RNA, aptamers, genes, plasmids, and derivatives or analogs thereof. In certain embodiments, the nucleic acid molecule is a microRNA.

Any suitable spacer group may be employed.

In certain embodiments, the co-polymer is a random co-polymer.

In certain embodiments, the co-polymer is a block co-polymer.

In certain preferred embodiments, the co-polymer is a block co-polymer: In certain preferred embodiments, the co-polymer comprises:

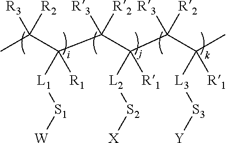

wherein each of i and j is independently a positive number, k may be zero or a positive number.

In certain embodiments, each of i and j is independently selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, k is 0. In certain embodiments, k is selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, each of R₂, R′₂, R″₂, R₃, R′₃ and R″₃ is a hydrogen, and each of R₁, R′₁ and R″₁ is a methyl group.

In certain embodiments, each of L₁, L₂ and L₃ is independently a

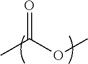

or an

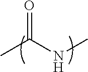

group.

In certain embodiments, W comprises

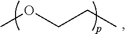

wherein p is an integer from about 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, W comprises

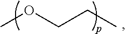

wherein p is an integer from about 1 to about 200.

In certain embodiments, W comprises a charged group. In certain embodiments, the charged group is selected from —NR₂ and —NR₃⁺, wherein R is hydrogen or a C₁-C₁₅ (e.g., C₁-C₁₂, C₁-C₉, C₁-C₆, C₁-C₃, C₃-C₁₅, C₆-C₁₅, C₉-C₁₅, C₃-C₉, C₆-C₁₂) alkyl group.

In certain embodiments, W is a zwitterionic group. In certain embodiments, the zwitterionic group is selected from the group consisting of:

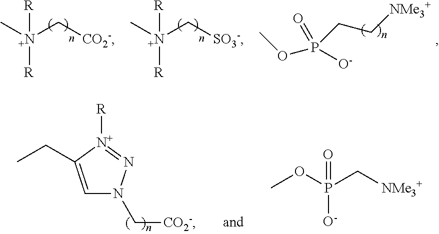

wherein each R is hydrogen or a C₁-C₁₅ (e.g., C₁-C₁₂, C₁-C₉, C₁-C₆, C₁-C₃, C₃-C₁₅, C₆-C₁₅, C₉-C₁₅, C₃-C₉, C₆-C₁₂) alkyl group; n is independently an integer from about 1 to about 12.

In certain embodiments, each n is independently 1. In certain embodiments, each n is independently an integer from about 2 to about 6 (e.g., 2, 3, 4, 5, 6).

In certain embodiments, W is a charge-neutral group. In certain preferred embodiments, the charge-neutral group is

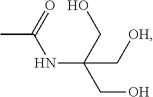

In certain embodiments, the polymer host comprises a network of a block or random co-polymer having the structural formula:

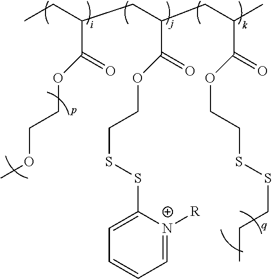

wherein each of p and q is independently an integer from about 1 to about 20 (e.g., from about 1 to about 15, from about 1 to about 12, from about 1 to about 9, from about 1 to about 6, from about 1 to about 3, from about 3 to about 15, from about 6 to about 15, from about 9 to about 15, from about 12 to about 15, from about 3 to about 12, from about 3 to about 9, from about 6 to about 9, from about 6 to about 12) and R is a C₁-C₁₅ (e.g., C₁-C₁₂, C₁-C₉, C₁-C₆, C₁-C₃, C₃-C₁₅, C₆-C₁₅, C₉-C₁₅, C₃-C₉, C₆-C₁₂) alkyl group.

In certain embodiments, the co-polymer is a random co-polymer.

In certain embodiments, the co-polymer is a block co-polymer.

In certain embodiments, X comprises a disulfide group.

In certain embodiments, X comprises a

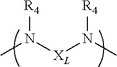

group, wherein each of R₄ and R′₄ is independently a hydrogen or C₁-C₁₂ (e.g., C₁-C₉, C₁-C₆, C₁-C₃, C₃-C₁₂, C₆-C₁₂, C₉-C₁₂, C₃-C₉, C₃-C₆) alkyl group and X_{L} is a spacer group.

In certain preferred embodiments, each of R₄ and R′₄ is hydrogen.

In certain embodiments, X_{L} is a pH-sensitive functional group.

In certain embodiments, the pH-sensitive functional group is

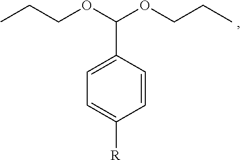

wherein R is hydrogen, a C₁-C₁₅ (e.g., C₁-C₁₂, C₁-C₉, C₁-C₆, C₁-C₃, C₃-C₁₅, C₆-C₁₅, C₉-C₁₅, C₃-C₉, C₆-C₁₂) alkyl group, or a

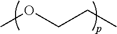

group, wherein p is about 1 to about 100 (e.g., from about 1 to about 50, from about 1 to about 30, from about 1 to about 20, from about 1 to about 10, from about 1 to about 6, from about 1 to about 3, from about 6 to about 100, from about 10 to about 100, from about 20 to about 100, from about 50 to about 100, from about 3 to about 20, from about 6 to about 20).

In certain embodiments, X_{L} is a peptide having from about 1 to about 20 (e.g., from about 1 to about 15, from about 1 to about 12, from about 1 to about 10, from about 1 to about 8, from about 1 to about 5, from about 1 to about 3, from about 3 to about 20, from about 5 to about 20, from about 10 to about 20, from about 15 to about 20, from about 3 to about 12, from about 3 to about 6, from about 6 to about 12) amino acid units that are cleavable by an enzyme.

In certain embodiments, Y is selected from a linear or branched C₁-C₂₀ (e.g., C₁-C₁₅, C₁-C₁₂, C₁-C₉, C₁-C₆, C₁-C₃, C₃-C₂₀, C₆-C₂₀, C₆-C₁₅, C₉-C₂₀, C₁₂-C₂₀, C₃-C₁₅, C₃-C₁₂, C₃-C₆, C₆-C_{12,}) alkyl group substituted with or without an aromatic moiety.

In certain embodiments, the crosslinked network of polymer molecules is crosslinked both inter-molecularly and intra-molecularly.

In certain embodiments, the crosslinked network of polymer molecules is crosslinked via disulfide bonds.

In certain embodiments, the crosslinked network of polymer molecules have a crosslinking density from about 1% to about 80%, relative to the total number of structural units in the polymer. In certain embodiments, the crosslinking density is from about 10% to about 60%, relative to the total number of structural units in the polymer. In certain embodiments, the crosslinking density is from about 10% to about 30%, relative to the total number of structural units in the polymer. In certain embodiments, the crosslinking density is from about 30% to about 60%, relative to the total number of structural units in the polymer.

In certain embodiments, the loading weight percentage of the nucleic acid is from about 0.2% to about 70% (e.g., from about 0.5% to about 70%, from about 2% to about 70%, from about 10% to about 70%, from about 0.2% to about 30%, from about 0.2% to about 10%, from about 0.2% to about 5%).

In certain embodiments, the de-crosslinking of the crosslinked polymer molecules is due to a biological or chemical stimulus at the biological site.

In certain embodiments, the stimulus is the redox environment at the biological site.

In certain embodiments, the stimulus is a pH value at the biological site.

In certain embodiments, the stimulus is an external light signal.

In certain embodiments, the biological site is within an organ or tissue of a subject. In certain embodiments, the biological site is inside a cell of a subject.

In certain embodiments, the nano-assembly has a diameter from about 3 nm to about 500 nm. In certain embodiments, the nano-assembly has a diameter from about 3 nm to about 20 nm. In certain embodiments, the nano-assembly has a diameter from about 20 nm to about 50 nm. In certain embodiments, the nano-assembly has a diameter from about 50 nm to about 100 nm. In certain embodiments, the nano-assembly has a diameter from about 100 nm to about 500 nm.

In certain embodiments, the nano-assembly is covalently linked to or non-covalently associated with a biological agent releasable at or near the biological site.

In another aspect, the invention generally relates to a block or random co-polymer, having the structural formula:

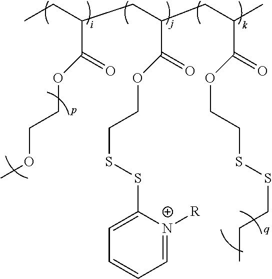

wherein
R is a C₁-C₁₅ alkyl group;
each of p and q is an integer from about 1 to about 20; and
each of i and j is independently a positive number, k may be zero or a positive number.

In certain embodiments, p is an integer selected from from about 1 to about 20 (e.g., from about 1 to about 15, from about 1 to about 12, from about 1 to about 10, from about 1 to about 8, from about 1 to about 5, from about 1 to about 3, from about 3 to about 20, from about 5 to about 20, from about 10 to about 20, from about 15 to about 20, from about 3 to about 12, from about 3 to about 6, from about 6 to about 12).

In certain embodiments, q is an integer selected from from about 1 to about 20 (e.g., from about 1 to about 15, from about 1 to about 12, from about 1 to about 10, from about 1 to about 8, from about 1 to about 5, from about 1 to about 3, from about 3 to about 20, from about 5 to about 20, from about 10 to about 20, from about 15 to about 20, from about 3 to about 12, from about 3 to about 6, from about 6 to about 12).

In certain embodiments, each of i and j is independently selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, k is 0. In certain embodiments, k is selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, the ratio of i:j is in the range from about 2:8 to about 8:2 (e.g., from about 3:7 to about 7:3, from about 4:6 to about 6:5, from about 1:1).

In certain embodiments, the co-polymer has a molecular weight from about 1,000 to about 100,000 (e.g., from about 1,000 to about 50,000, from about 1,000 to about 20,000, from about 1,000 to about 10,000, from about 5,000 to about 100,000, from about 10,000 to about 100,000, from about 20,000 to about 100,000, from about 50,000 to about 100,000).

In yet another aspect, the invention generally relates to a method for delivering a nucleic acid molecule. The method includes: forming a crosslinked polymeric nanogel-nucleic acid assembly comprising a crosslinked polymeric nanogel and entrapped nucleic acid molecules therein, wherein the crosslinked polymeric nanogel is characterized by a polymeric network that is partially or completely free of cationic moieties; and directing the crosslinked polymeric nanogel-nucleic acid assembly to a target site.

In certain embodiments, the method further includes releasing the entrapped nucleic acid molecules at the target site.

In certain embodiments of the method, forming a crosslinked polymeric nanogel-nucleic acid assembly includes: providing a polymer comprising one or more cationic moieties, wherein the polymer comprises one or more crosslinking groups; forming an electrostatic complex between the polymer and nucleic acid molecules; crosslinking the polymer chains to release one or more cationic moieties and form a polymeric network with the nucleic acid molecule entrapped therein.

In certain embodiments of the method, the nucleic acid molecule is selected from single-stranded or double-stranded RNA or DNA, and derivatives or analogs thereof.

In certain embodiments of the method, the nucleic acid molecule is selected from the group consisting of dsRNA, siRNA, mRNA, ncRNA, microRNA, catalytic RNA, guide RNA, aptamers, genes, plasmids, and derivatives or analogs thereof.

In certain embodiments of the method, the polymer is a random or block co-polymer.

In certain embodiments of the method, the polymeric nanogel comprises a block or random co-polymer comprising structural units of:

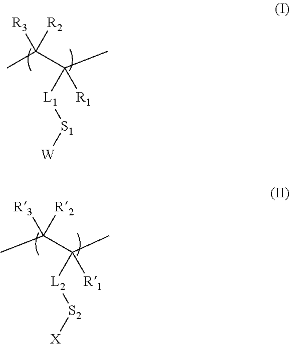

wherein
each of R₁ and R′₁ is independently a hydrogen, C₁-C₁₂ alkyl group, or halogen;
each of R₂, R′₂, R₃, and R′₃ is independently a hydrogen, (C₁-C₁₆) alkyl, (C₁-C₁₆) alkyloxy, or halogen;
each of L₁ and L₂ is independently a linking group;
each of S₁ and S₂ is independently a single bond or a spacer group;
W is a hydrophilic group; and
X is a group comprising a crosslinking moiety.

In certain embodiments of the method, the polymeric nanogel comprises a block or random co-polymer having the structural formula:

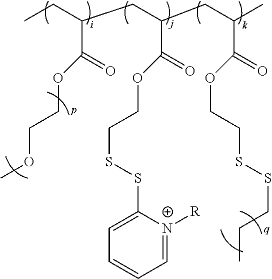

wherein
R is a C₁-C₁₅ alkyl group;
each of p and q is an integer from about 1 to about 20; and
each of i and j is independently a positive number, k may be zero or a positive number.

Each of p and q is an integer selected from from about 1 to about 20 (e.g., from about 1 to about 15, from about 1 to about 12, from about 1 to about 10, from about 1 to about 8, from about 1 to about 5, from about 1 to about 3, from about 3 to about 20, from about 5 to about 20, from about 10 to about 20, from about 15 to about 20, from about 3 to about 12, from about 3 to about 6, from about 6 to about 12).

In certain embodiments, each of i and j is independently selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, k is 0. In certain embodiments, k is selected from 1 to about 500 (e.g., from about 1 to about 500, from about 1 to about 300, from about 1 to about 200, from about 1 to about 100, from about 1 to about 50, from about 1 to about 20, from about 1 to about 10, from about 10 to about 500, from about 50 to about 500, from about 100 to about 500, from about 200 to about 500, from about 10 to about 100, from about 10 to about 50, from about 10 to about 20, from about 20 to about 200, from about 20 to about 100).

In certain embodiments, the ratio of i:j is in the range from about 2:8 to about 8:2 (e.g., from about 3:7 to about 7:3, from about 4:6 to about 6:5, from about 1:1).

In certain embodiments, the co-polymer has a molecular weight from about 1,000 to about 100,000 (e.g., from about 1,000 to about 50,000, from about 1,000 to about 20,000, from about 1,000 to about 10,000, from about 5,000 to about 100,000, from about 10,000 to about 100,000, from about 20,000 to about 100,000, from about 50,000 to about 100,000).

### Synthesis of Methylated PDS-PEG Copolymers and Crosslinked Complexes

### Monomer Synthesis

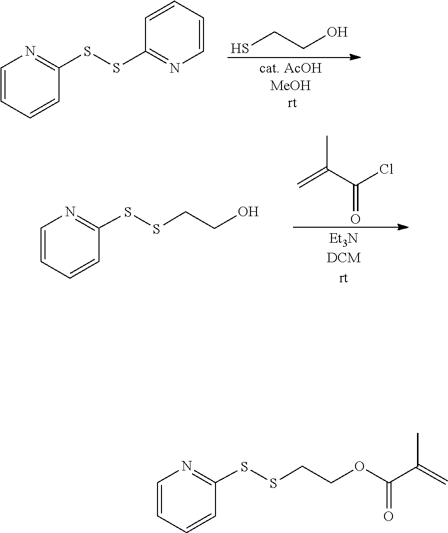

The two steps were done according to the previous report (Macromolecules 2006, 39, 5595-5597.) with 87% and 93% yield, respectively.

### Polymer Synthesis

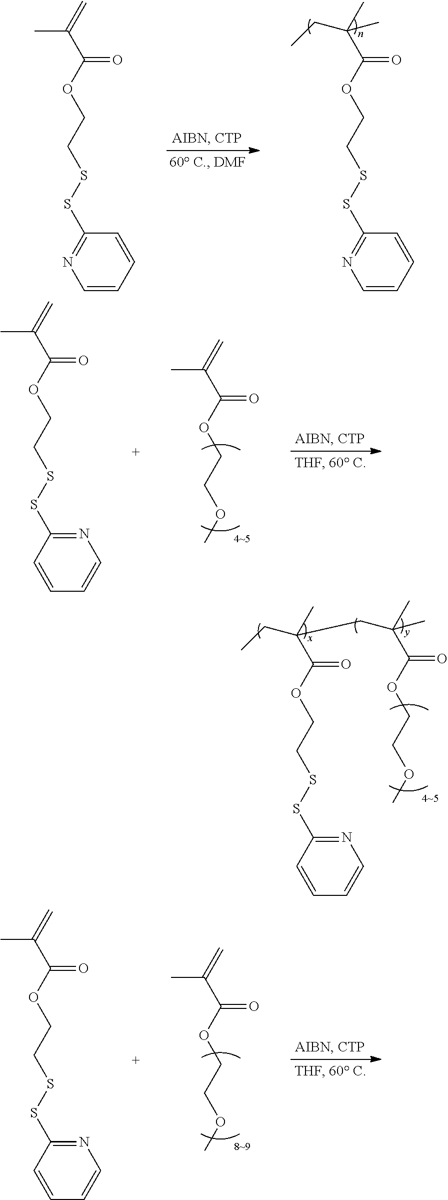

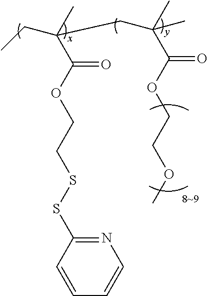

The polymerization reactions were performed according to a previous report (J. Am. Chem. Soc. 2010, 132, 8246-8247.). The homopolymer was used to check the proper conditions of polymer methylation. Three PEG-PDS copolymers were synthesized: P2, x:y=0.43:0.57, the average molecular weight of PEG is 300 g·mol⁻¹; P3, x:y=0.66:0.34, the average molecular weight of PEG is 500 g·mol⁻¹; P4, x:y=0.88:0.12, the average molecular weight of PEG is 500 g·mol⁻¹.

### Methylation of PEG-PDS Copolymers

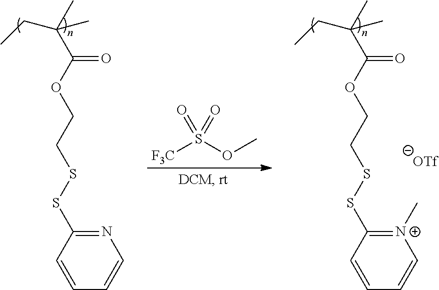

The procedure of methylation using methyl trifluoromethanesulfonate was adapted from a previous report (Organometallics 2010, 29, 5821-5833.). Generally, 1.1 equiv. of methyl trifluoromethanesulfonate was added to the dichloromethane solution of PDS homopolymer (or PEG-PDS copolymer). For example, P2 (993 mg) was dissolved in 10 mL dichloromethane. Methyl trifluoromethanesulfonate (638 mg) was added into the solution in one portion. After stirring for 2 hrs at room temperature, the mixture was washed with diethyl ether for three times. The complete methylation is confirmed by the aromatic proton shift and the addition of the methyl group at δ4.4.

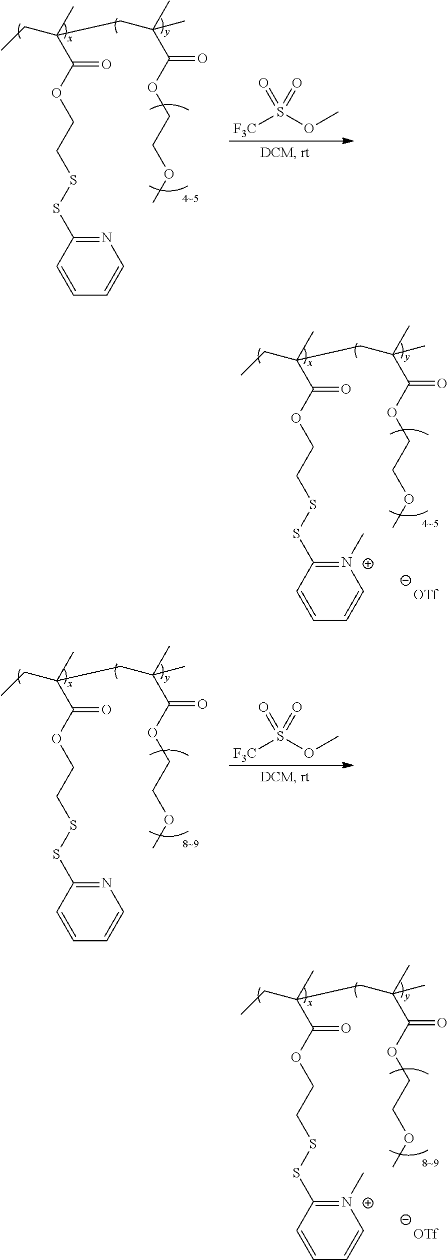

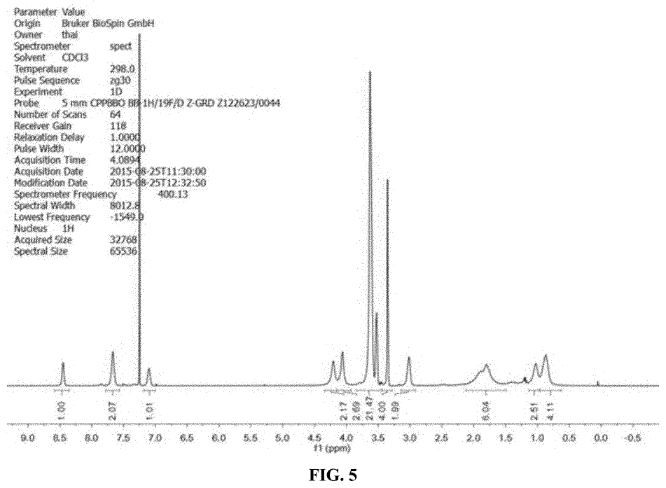

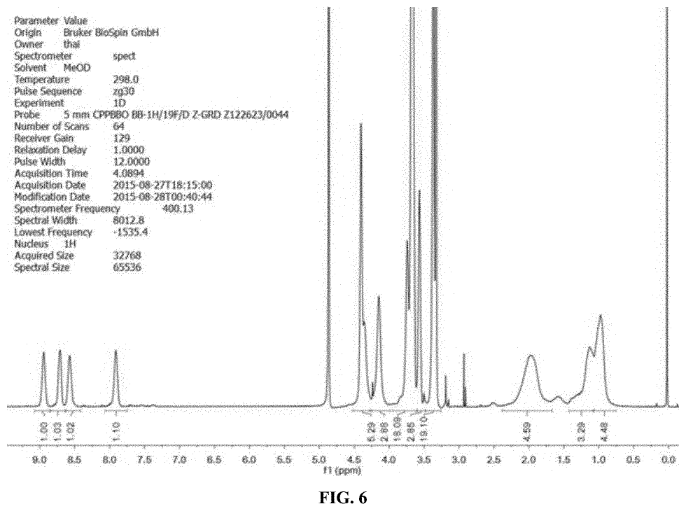

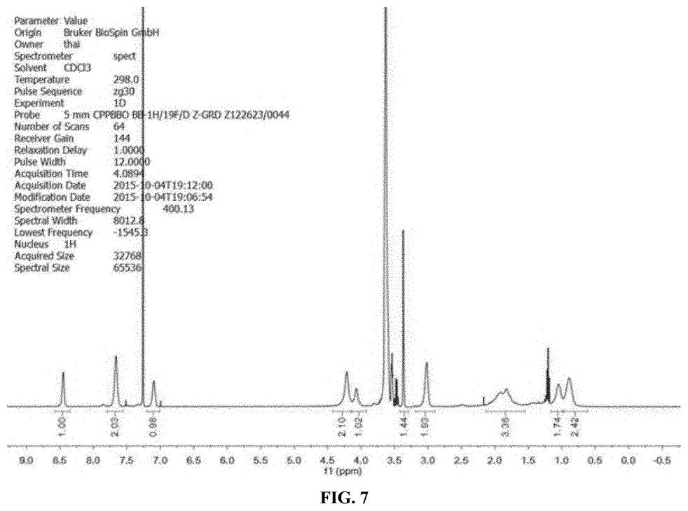

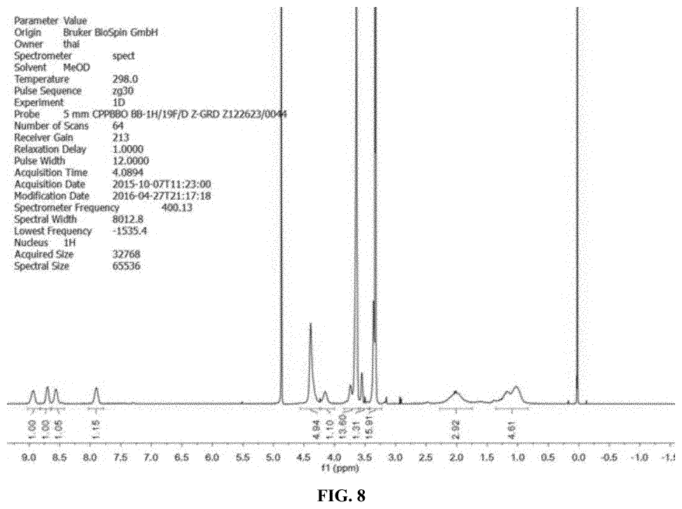

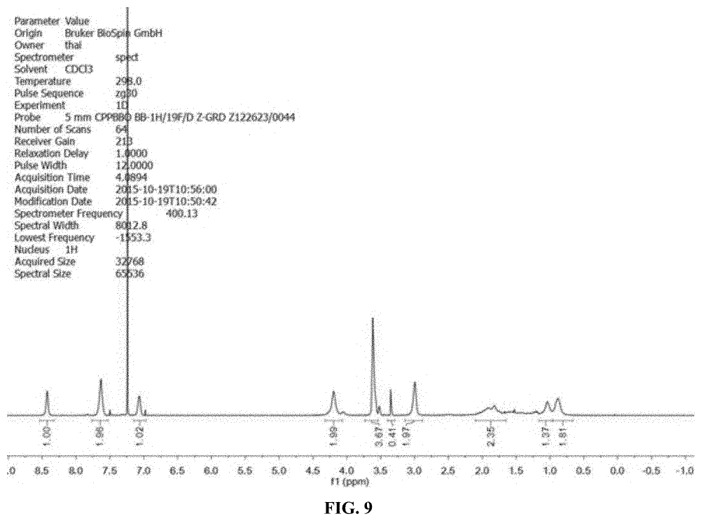

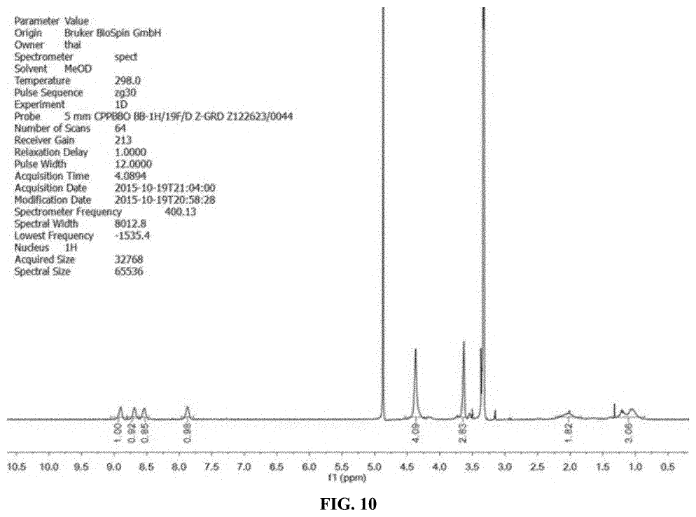

The methylation was characterized by ¹H NMR. FIGS. 5-10 are ¹H NMR spectrum of P2, methylated P2, P3, methylated P3, P4, methylated P4.

Synthesis of Crosslinked dsRNA-methylated Polymer Complex

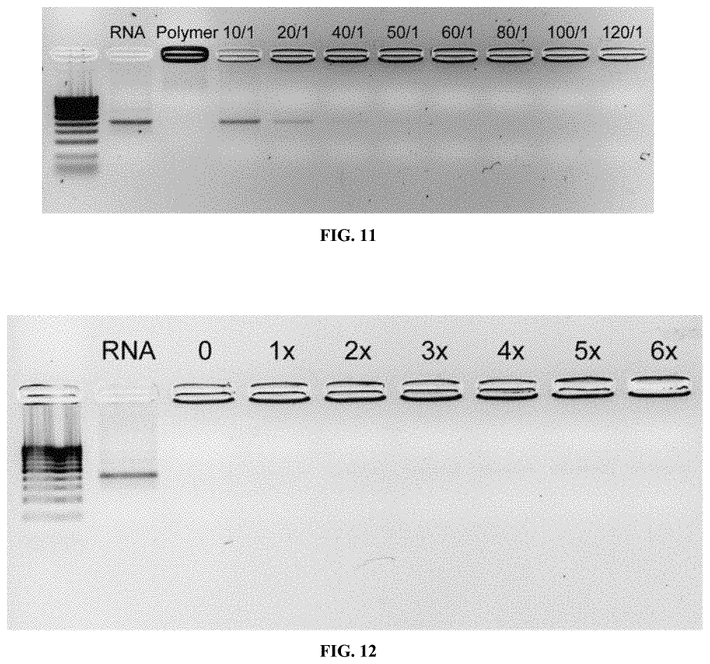

The experiments were carried out in phosphate buffer (pH=7.4) solution. A pre-optimized N/P ratio is required to be obtained before the DTT-induced crosslinking. To determine the N/P ratio, the dsRNA amount was kept constant at 100 ng per sample and incubated with an increasing amount of methylated polymers. The optimal ratios for methylated P2, P3, and P4 are 900/1, 800/1, and 40/1, respectively. FIG. 11 shows the agarose gel electrophoresis result of methylated P4.

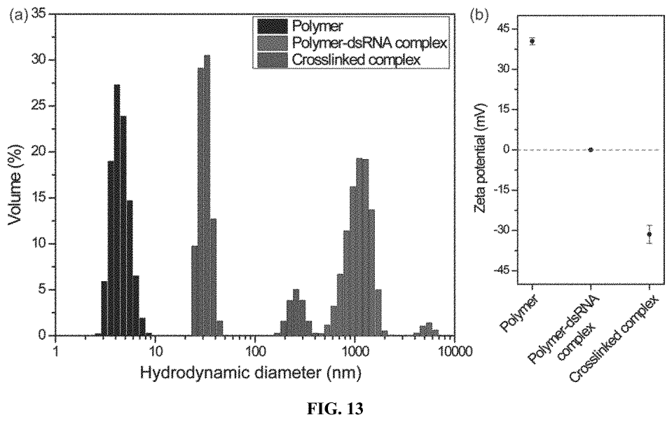

DTT-induced crosslinking of polymer-RNA complex. The amount of polymer in each well was 5.28 μg. 0, 1x, 2x, 3x, 4x, 5x, 6x represent the varied amount of DTT, where x=158 ng. 6x is the calculated amount for the complete crosslinking of polymer. Nanogel represents the DTT-crosslinked polymer. No leakage from the complex was observed DTT-induced during crosslinking. FIG. 12 shows the DTT-induced crosslinking result.

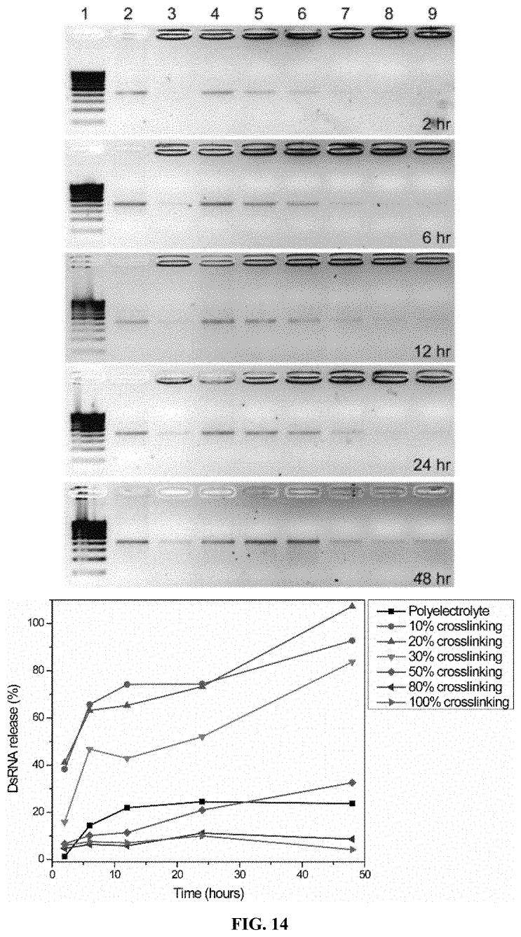

Methylated P4 was further characterized by dynamic light scattering and zeta potential measurement. (FIG. 13)

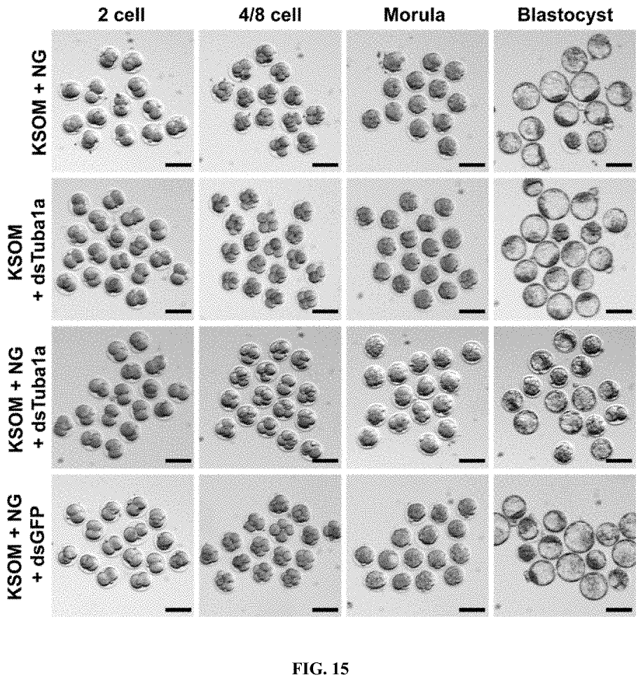

The complexes with different crosslinking percentage were evaluated in presence of glutathione. A tunable dsRNA release behavior was observed. In FIG. 14, 2 is the dsRNA control sample; 3 is the RNA-polymer complex; 4˜9 are the complexes with different crosslinking density: 4=10%, 5=20%, 6=30%, 7=50%, 8=80%, 9=100%.

### Blastocyst Development

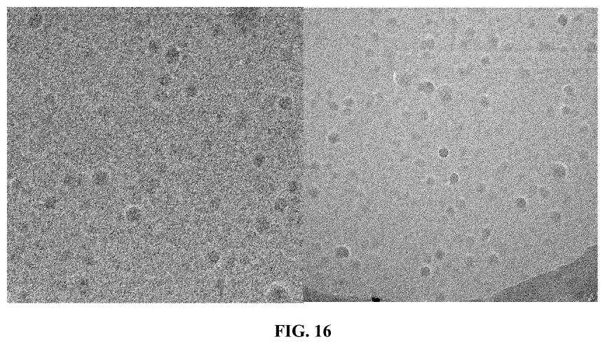

Blastocyst development monitored at different preimplantation stages is shown in FIG. 15. NG represents the crosslinked polymer. “NG+dsTuba1a” represents the crosslinked polymer-dsTuba1a complex. “NG+dsGFP” represents the crosslinked polymer-dsGFP complex. The scale bar in each figure represents 100 μm.

### Cryogenic Electron Microscopy (CryoEM)

Cryo-EM was performed on a FEI Sphera microscope operating at 200 keV. CryoEM grids were prepared by depositing 4 μL of sample onto a Quantifoil R2/2 TEM grid that had previously been glow discharged using an Emitech K350 glow discharge unit and plasma-cleaned for 90 s in an E.A. Fischione 1020 unit. The grids were blotted with filter paper under high humidity to create thin films, then rapidly plunged into liquid ethane. The grids were transferred to the microscope under liquid nitrogen and kept at <−175° C. while imaging. Micrographs were recorded on a 2k by 2k Gatan CCD camera. The images below show that the particle size correspond to those obtained with dynamic light scattering measurements. The images are shown in FIG. 16.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1-49. (canceled)

50. A method for forming a nucleic acid-containing crosslinked polymeric nanogel assembly, comprising:
providing a polymer comprising one or more cationic moieties, wherein the polymer comprises one or more crosslinking groups;
forming an electrostatic complex between the polymer and a nucleic acid molecule; and
crosslinking the polymer to release one or more cationic moieties and to form a nucleic acid-containing polymeric nanogel assembly with the nucleic acid molecule entrapped in the crosslinked polymeric nanogel,
wherein
the polymer comprises a block or random co-polymer having the structural formula: 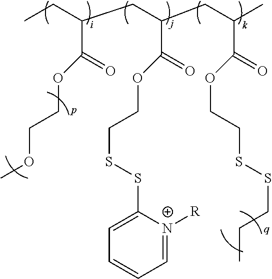
wherein
R is a C₁-C₁₅ alkyl group;
each of p and q is an integer from about 1 to about 20; and
each of i and j is independently a positive number, k may be zero or a positive number.

51. The method of claim 50, wherein the nucleic acid molecule is selected from the group consisting of single-stranded or double-stranded RNA or DNA, and derivatives or analogs thereof.

52. The method of claim 51, wherein the nucleic acid molecule is selected from the group consisting of dsRNA, siRNA, mRNA, ncRNA, microRNA, catalytic RNA, guide RNA, aptamers, genes, plasmids, and derivatives or analogs thereof.

53-55. (canceled)

56. The method of claim 50, further comprising:
directing the crosslinked polymeric nanogel-nucleic acid assembly to a target site; and
releasing the entrapped nucleic acid molecules at the target site.

57-63. (canceled)
